(19) United States
(12) Patent Application Publication
(10) Pub. No.: US 2025/0075239 A1
(43) Pub. Date: Mar. 6, 2025

(54) ENZYMATIC DNA SYNTHESIZER DEVICE

(71) Applicant: Lenovo (Singapore) Pte, Ltd., Singapore (SG)

(72) Inventors: Vanessa Pereira Gomes, Butanta (BR); Rodrigo Anjos de Souza, Butanta (BR); Gabriel Griep, Butanta (BR); Bruno Nobuya Katayama Gobara, Butanta (BR); Joao Lucas Maehara Said dos Reis, Butanta (BR); Eric Andre Velasco, Butanta (BR); Henrique da Costa Oliveira, Sao Paulo (BR); Bruno Marinaro Verona, Butanta (BR); Natalia Neto Pereira Cerize, Sao Paulo (BR)

(21) Appl. No.: 18/459,271

(22) Filed: Aug. 31, 2023

(51) Int. Cl. C12P 19/34; B01J 19/00; C12N 15/10; G16B 50/30

(52) U.S. Cl. CPC C12P 19/34; B01J 19/0046; C12N 15/10; G16B 50/30; B01J 2219/00497; B01J 2219/00759

(57) ABSTRACT

A microfluidic system for performing nucleic acid synthesis includes a microfluidic plate having a reaction chamber coupled to a microfluidic plate input and a microfluidic plate output. A temperature control plate is thermally coupled to the microfluidic plate. A reagent injection plate is coupled to receive enzymatic synthesis reagents. A microvalve plate is coupled between the reagent injection plate and the microfluidic plate input. A controller is coupled the temperature control plate, and the microvalve plate to control the microfluidic system to controllably synthesize nucleic acid sequences.

### BACKGROUND

Challenges with utilizing DNA for data storage are cost and efficiency. The task of encoding data can be very slow, with some rates being about 400 bytes per second. Synthesizing DNA for data storage has been performed utilizing phosphoramidite based chemical processes which are performed using large equipment. Such equipment can be quite costly, making DNA synthesis quite capital intensive, slow, and impractical to scale to meet current and future data storage requirements.

### SUMMARY

A microfluidic system for performing nucleic acid synthesis includes a microfluidic plate having a reaction chamber coupled to a microfluidic plate input and a microfluidic plate output. A temperature control plate is thermally coupled to the microfluidic plate. A reagent injection plate is coupled to receive enzymatic synthesis reagents. A microvalve plate is coupled between the reagent injection plate and the microfluidic plate input. A controller is coupled the temperature control plate, and the microvalve plate to control the microfluidic system to controllably synthesize nucleic acid sequences.

### BRIEF DESCRIPTION OF THE DRAWINGS

FIG. 1A is a block diagram schematic of a microfluidic based enzymatic synthesizer for synthesizing data encoded DNA according to an example embodiment.

FIG. 1B is block diagram representation of an example microparticle bead according to an example embodiment.

FIG. 2A is a perspective view of an example synthesizer system according to an example embodiment.

FIG. 2B is a top view of an elliptical reaction chamber according to an example embodiment.

FIG. 2C is a side cross section view of the reaction chamber taken along lines 2C-2C of FIG. 2B according to an example embodiment.

FIG. 2D is a perspective view of the reaction chamber according to an example embodiment.

FIG. 3 is a side view 300 of a synthesizer system illustrating multiple layers of channels and the vertical serpentine nature of a mixer according to an example embodiment.

FIG. 4 is a block schematic diagram of a filter illustrating dimensions of a comb structure having openings according to an example embodiment.

FIG. 5 is a block schematic diagram of filter illustrating dimensions of a comb structure having openings according to an example embodiment.

FIG. 6 is a block cross section diagram of a microfluidic plate of an enzymatic synthesizer according to an example embodiment.

FIG. 7 is an example of positions of temperatures sensors and heaters on circuitry in a synthesizer according to an example embodiment.

FIG. 8 is a cross section representation of an example synthesizer system according to an example embodiment.

FIG. 9 is a flowchart illustrating a method of synthesizing nucleic acid sequences utilizing a microfluidic synthesizer system according to an example embodiment.

FIG. 10 is a block schematic diagram of a computer system to implement one or more example methods.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The advent of digital computing in the 20^{th} Century created the need for archival storage of large amounts of digital or binary data. Archival storage is intended to house data for long periods of time, e.g., years, decades or longer, in a way that is very low cost, and that supports the rare need to re-access the data. Although an archival storage system may feature the ability to hold unlimited amounts of data at very low cost, such as through a physical storage medium able to remain dormant for long periods of time, the data writing and recovery in such a system can be the relatively slow or otherwise costly processes. The dominant forms of archival digital data storage that have been developed to date include magnetic tape, and, more recently, compact optical disc (CD). However, as data production grows, there is a need for even higher density, lower cost, and longer lasting archival digital data storage systems.

It has been observed that in biology, the genomic DNA of living organisms functions as a form of digital information archival storage. On the timescale of the existence of a species, which may extend for thousands to millions of years, the genomic DNA in effect stores the genetic biological information that defines the species. The complex enzymatic, biochemical processes embodied in the biology, reproduction and survival of the species provide the means of writing, reading and maintaining this information archive. This observation has motivated the idea that perhaps the fundamental information storage capacity of DNA could be harnessed as the basis for high density, long duration archival storage of more general forms of digital information.

What makes DNA attractive for information storage is the extremely high information density resulting from molecular scale storage of information. In theory for example, all human-produced digital information recorded to date, estimated to be approximately 1 ZB (ZettaByte) (^{˜}10²¹ Bytes), could be recorded in less than 10²² DNA bases, or 1/60^{th} of a mole of DNA bases, which would have a mass of just 10 grams. In addition to high data density, DNA is also a very stable molecule, which can readily last for thousands of years without substantial damage, and which could potentially last far longer, for tens of thousands of years, or even millions of years, such as observed naturally with DNA frozen in permafrost or encased in amber.

Despite the great capacity and stability of DNA based data storage, both the cost of synthesizing data encoded DNA, and the relative slowness of such encoding and reading, have been obstacles to using DNA for more than experimental purposes. Machines used in the traditional DNA synthesis by phosphoramidite chemistry technology are extensive, capital-intensive, and slow.

A miniaturized enzymatic DNA synthesizer device provides significant advantages to improve the DNA synthesis. The enzymatic DNA synthesizer facilitates an automated synthesis process that provides greater synthesis accuracy and speed than traditional methods, while reducing environmental impact by minimizing the use of harsh chemicals. The device's miniaturization and process automation could extend DNA synthesis beyond labs, taking solutions to areas such as medicine, agriculture and DNA data storage.

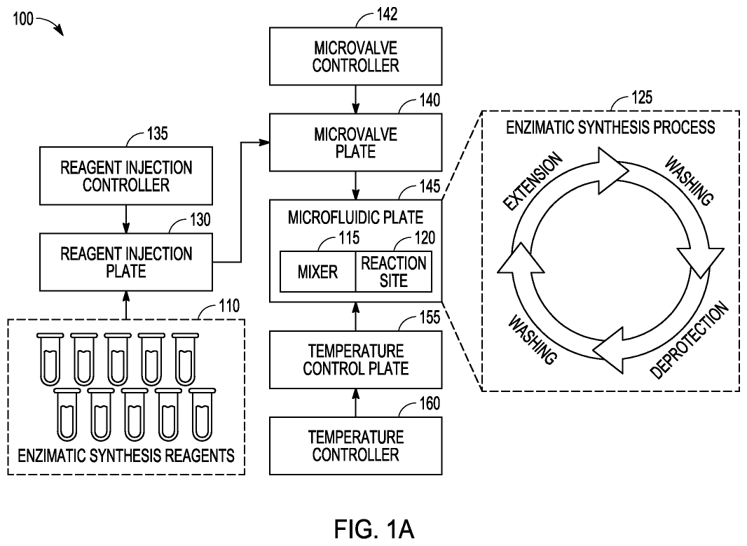

FIG. 1A is a block diagram schematic of a microfluidic based enzymatic synthesizer 100 for synthesizing data encoded DNA. Synthesizer 100 utilizes enzymatic synthesis reagents 110 that are selectively provided through a mixer 115 to a reaction chamber 120 that is temperature controlled to perform an enzymatic synthesis process 125. The reagents may include enzymes and monomers in one example, as well as washing fluid. The synthesizer 100 is responsible for mixing reagents 110 and controlling their supply to the reaction chamber 120 for controlled DNA synthesis following general steps of extension, washing, deprotection, and washing shown in process 125.

The synthesizer 100 is much smaller than current systems and lends itself to parallelization, which can greatly increase the rate of data encoded DNA synthesis. The ability to control the temperature of the reaction chamber at a microfluidic size facilitates much faster temperature changes, allowing much faster DNA synthesis than prior systems.

A reagent injection plate 130 may be controlled by an injection controller 135 to control which reagents are provided to the reaction chamber 120. In one example, a microvalve plate 140 may be coupled to the reagent injection plate controlled by a microvalve controller 142 to provide selected reagents for synthesis of a corresponding nucleotide base, such as one of adenine (A), cytosine (C), guanine (G) and thymine (T). Different combinations of reagents may be used to add each such nucleotide base, with the process 125 being performed for each added nucleotide base.

The reaction chamber 120 may be formed on a microfluidic plate 145 that is coupled to receive the reagents from the microvalve plate 140. The microfluidic plate 145 may include the mixer 115 disposed between the microvalve plate 140 and the reaction chamber 120 to ensure the reagents are mixed well prior to entering the chamber. Mixer 115 receives the enzymatic reagents that leave the injection plate 130 and pass through inlet channels with the action of the microvalves in microvalve plate 140. A serpentine construction of a channel in the mixer 115 causes turbulence of the fluid, which is desirable to mix the reagents.

In one example, the microfluidic plate 145 is a microfluidic subsystem. The microfluidic plate 145 and other plates of the synthesizer 100 may be made of biocompatible materials suitable for the purpose of the respective components. The plates in one example are constructed of a glass ceramic substrate, such as a Low Temperature Co-Fired Ceramic (LTCC) forming a multilayer microfluidic device that may have multiple reaction chambers 120 for scalable enzymatic manufacturing of genetic material and/or nucleic acid for data storage applications. Other suitable materials include polymers such as cyclic olefin copolymer (COC) which is an amorphous polymer, polypropylene, polycarbonate, or polyethylene provided they can maintain integrity at desired process temperatures and in light of various reagents utilized.

In some examples, the shape of the reaction chamber 120 may be designed to facilitate uniform distribution of the mixed reagents. In one example, the chamber is oval or elliptical in shape. The reaction chamber 120 may also be concave or deeper near a middle of the reaction chamber 120, such that gravity helps congregate microparticle beads, also referred to as initiator particles toward the middle of the reaction chamber 120. The middle is thus deeper than edges or a perimeter of the reaction chamber 120. Both modifications, alone or together, can help to distribute the reagents and reaction sites toward a middle portion of the chamber where reactions to form the nucleotide bases occur.

FIG. 1B is a block diagram representation of an example microparticle bead 170. Bead 170 includes a core 171 and a substrate 172. The core may be any suitable material, such as glass, for supporting the substrate 172. The substrate 172 or surface of the bead 170 can include silver, gold, glass, iron, or a combination thereof. Each bead also includes a linker molecule 175 functionalized to the substrate. Each bead also includes an initiator oligonucleotide sequence 180 functionalized to the linker molecule. Each bead can have any shape, respecting the fact that it is smaller than the inlet and larger than the filter inside the reaction site.

The microfluidic plate 145 may also be thermally coupled to a temperature control plate 155. The temperature control plate 155 may include heating elements, such as resistive heating elements that are controlled to desired temperatures by a temperature controller 160 that operates in conjunction with the reagent injection controller to help perform the synthesis process 125. Controller 160 may implement PID based control to vary and control the temperature to selected setpoints for one or more stages of processing where temperature facilitates processing.

The synthesis process 125 may include several steps in a cyclical manner, including washing, extension, washing and deprotection. Multiple of these steps are performed at different temperatures. The temperature control plate and controller operate as a temperature control subsystem that actuates and monitors the current reaction site temperature. When temperature of the reaction chamber is brought up to the enzymes' optimal operating temperature, the DNA synthesis reaction occurs. In some examples, the temperature control subsystem and reagent control operate to time the provision of reagents into the chamber at or near the time that such optimal operating temperatures are reached.

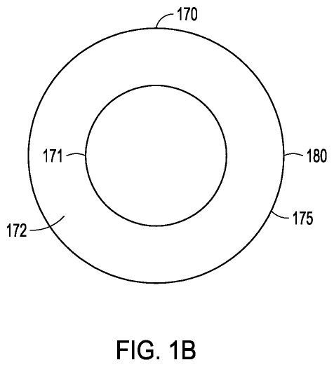

FIG. 2A is a perspective view of an example synthesizer system 200 based on the block diagram schematic of FIG. 1 with like reference numbers for like components. The reagents may enter a microfluidic supply chamber or chambers indicated at 210 and initiator particles may enter at chamber 215. Both reagents and initiator particles may be pumped through respective channels 220, 225 incoming from the reagent injection plate 130.

Microvalve plate 140 is shown connecting the channels 220, 225 to channels 230, 235 in the microfluidic plate 145. The channels 230, 235 are coupled to mixer 115 which comprises multiple layers of connected T-shaped channels in one example to facilitate reagent mixing. The T-shaped channels comprise a main channel having a varying depth as shown in a later cross section, and multiple orthogonal short channels, in which vortex type flows are promoted to further facilitate mixing.

The orthogonal short channels may comprise opposing pairs of short channels 240 (example width of 0.30 mm) coupled by short channels having a length of 0.30 mm, with the first few such pairs being longer (2.36 mm) than succeeding pairs. In one example, three long pairs of orthogonal channels 242 are followed by four or more shorter pairs (1.33 mm) of orthogonal channels 244 before a final channel 245 couples the mixer 115 to the portion 250 of the microfluidic plate containing the reaction chamber.

Portion 250 which includes the reaction chamber, may be formed with multiple layers of LTCC in one example. The height of the reaction chamber in one example is 1.96 mm (which is equivalent to seven layers of 265 um thick LTCC and two layers of 50 um thick LTCC. In one example method, each layer is cut using a laser. The cut LTCC layers may be in the form of a tape, and are stacked together and laminated, with controlled temperature and pressure to form the microfluidic plate. Subsequently, the microfluidic plate is sintered in an oxidizing atmosphere at 850° C. following a pre-established heating ramp. Glass paste or other adhesive may be used to bond the plate to other materials, such as PDMS. In further examples, the use of highly polished surfaces may provide sufficient binding.

The reaction chamber 120 may include two radially spaced outlets, first outlet 255 and second outlet 260, each with optional corresponding filters 265 and 270. In one example, second outlet 260 is coupled to a first outlet channel 272. The outlets 255 and 260 via channel 272 are coupled to an output channel 280 and collection chamber 282 from which data encoded DNA may be extracted and stored. Outlet channel 272 may have a width of 0.41 mm and output channel 280 has a width of 0.50 mm in one example.

The use of two outlets 255 and 260 containing filters helps to retain fluid outflow and improves fluid distribution inside the reaction chamber 120 while also preventing particles containing DNA from being washed out. For volumetric flow of 0.5 ml/min at the inlet, vortex formation within the reaction chamber favors the creation of reaction particulates near a center of the reaction chamber. Drainage through the filters reduces clogging, allowing synthesized DNA to exit the chamber after the conclusion of the enzymatic synthesis process.

FIG. 2B is a top view of the reaction chamber 120 showing the elliptical shape of the reaction chamber 120 in one example. Also shown are channel 245, first outlet 255, and second outlet 260. A length 284 of the reaction chamber 120, extending between channel 245, which serves as an input, and first outlet 255 is longer than the width 286 of the reaction chamber 120.

FIG. 2C is a side cross section view of the chamber taken along lines 2C-2C of the reaction chamber 120 shown in FIG. 2B. Multiple layers are shown at 287, 288, 289, 290, 291, 292, 293, and 294. Conceptually, the layers in FIG. 2C represent open space in the reaction chamber 120. In various examples, layers 288, 289, 290, 291, and 292 may have a thickness of 0.27 mm and a width of 4.48 mm. Layers 293 and 294 form a bottom portion of the reaction chamber 120 and have thicknesses of 0.05 mm. Layer 293 has a width of 2.59 mm and layer 294 has a width of 1.72 mm as shown. Layer 287 may be thicker in some examples, and defines a lateral extent of the reaction chamber. Layers 288, 289, 290, 291, and 292 are wider, and represent the open volume of the second outlet 260. The user of layers 291 and 292 which decrease in width, help concentrate microparticle beads near a middle of the reaction chamber with the assistance of gravity or centrifugal force in some examples.

FIG. 2D is a perspective view of the reaction chamber 120 illustrating the overall shape of the reaction chamber having a shallower center portion.

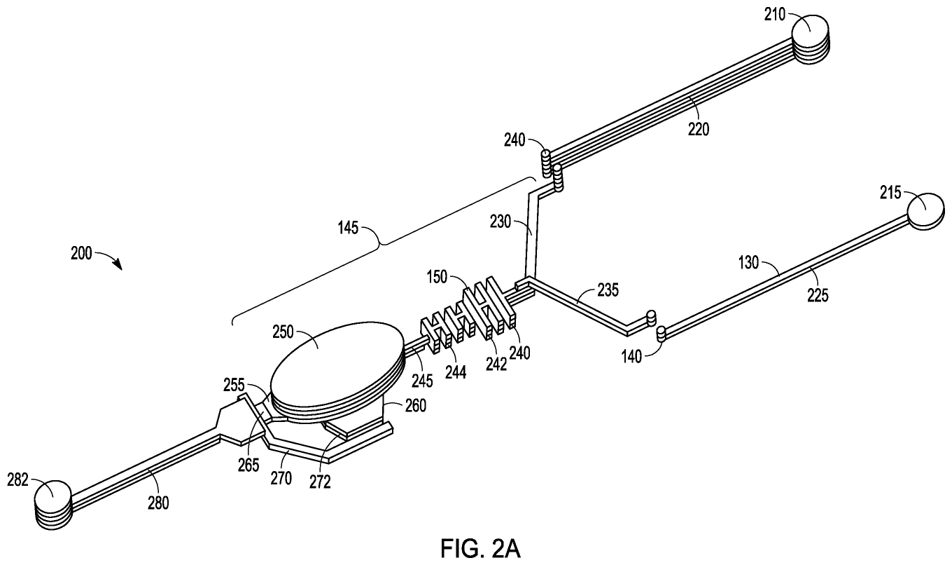

FIG. 3 is a side view 300 of the synthesizer system 200 illustrating multiple layers of channels and the vertical serpentine nature of the mixer 115. Mixer 115 serpentines with vertical channels 310 coupling horizontal channels 315 providing a vertical and meandering path along the progression of the mixer 115 towards the reaction chamber 120.

Temperature control plate 155 is disposed beneath and in thermal contact with plate material forming reaction chamber 120 in a vertically stacked manner to facilitate heat transfer and better temperature control of reaction chamber 120. In one example temperature control plate 155 may be a 50 um thick layer having a smaller area that the reaction chamber 120.

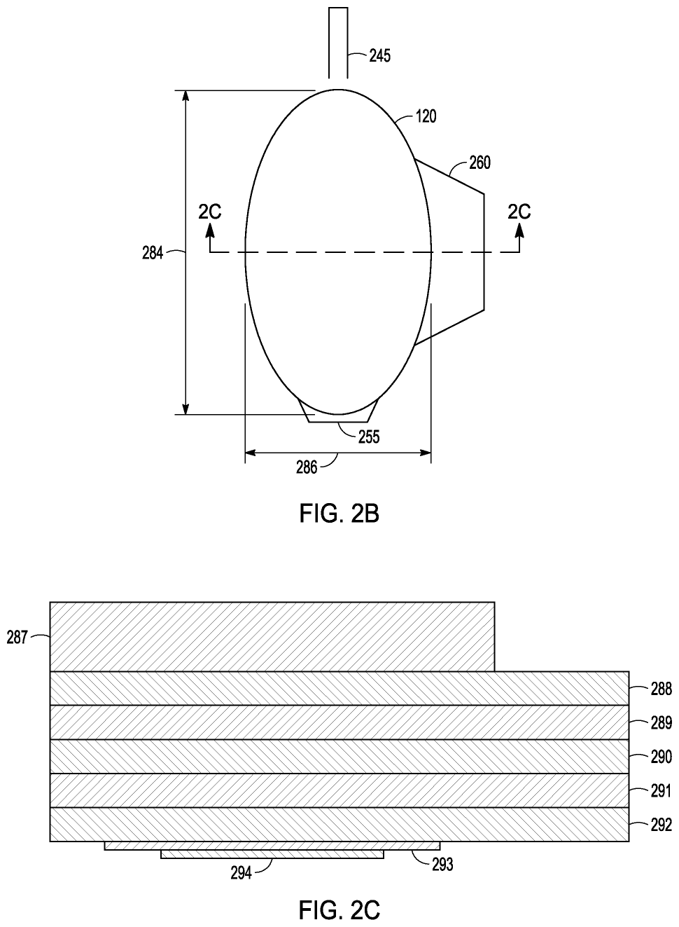

FIG. 4 is a block schematic diagram of filter 265 illustrating dimensions of a comb structure having openings 410 of approximately 0.02 mm and a spacing 415 of 0.08 mm.

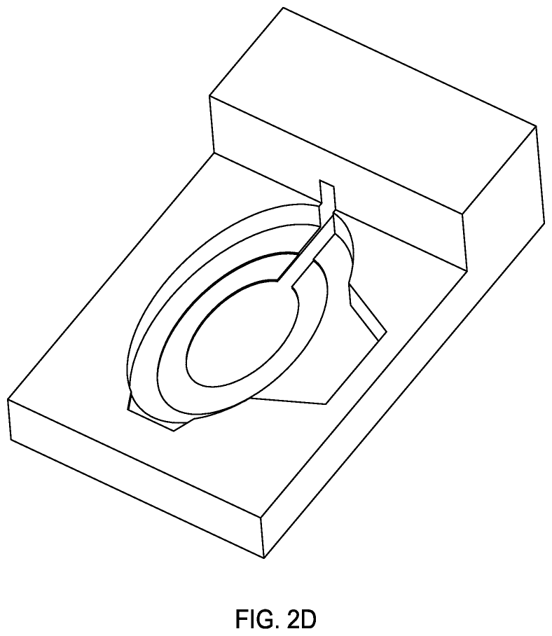

FIG. 5 is a block schematic diagram of filter 270 illustrating dimensions of a comb structure having openings 510 of approximately 0.02 mm and a spacing 515 of 0.08 mm. The filter openings are smaller in size than the initiator particles, allowing more process steps while the initiator particles remain in the reaction chamber 120.

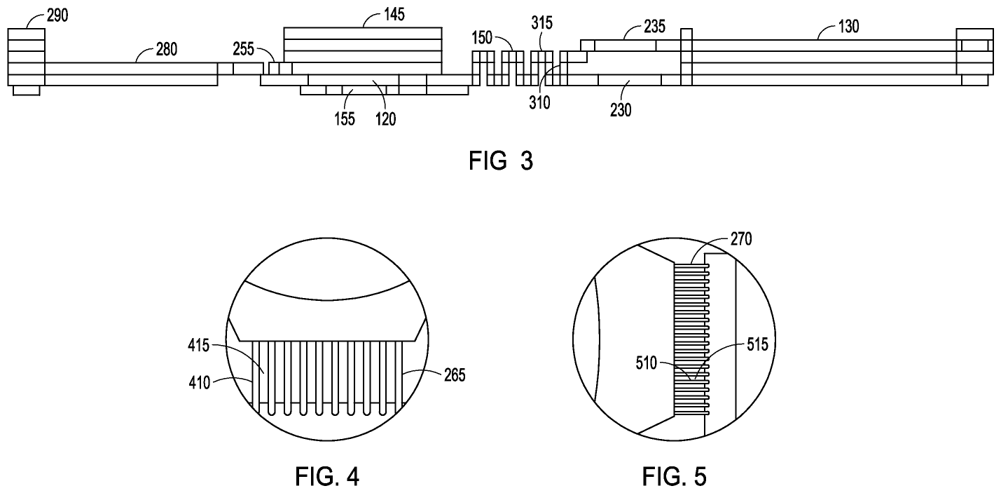

FIG. 6 is a block cross section diagram of the microfluidic plate 145 containing the reaction chamber 120. In one example, the microfluidic plate 145 may be secured to a substrate 610 that is heat conductive. Aluminum may be used for the substrate 610 in one example, and the microfluidic plate 145 may be screwed to the substrate 610 to position the microfluidic plate 145 in a desired portion of the substrate 610.

An inlet 615 may provide reagent to the reaction chamber 120. In one example, a valve 620 may be placed in a channel 625 between the inlet 615 and the reaction chamber 120 to control fluid flow into and out of the reaction chamber via channel 630 and outlet 635.

The substrate 610 may be supported on a printed circuit board (PCB) 650 that may include circuitry 640, 645 on either side for providing sensors and controllers for operation of the synthesizer system. Circuitry 645 may include one or more resistive heaters for heating the substrate 610 and by heat conduction, the microfluidic plate 145 and reaction chamber 120 formed therein.

Temperature sensors in the circuitry 645 or circuitry 640 will sense the temperature of the substrate 610 which should be nearly the same as the temperature in the reaction chamber 120, which should be centered on the substrate 610 with respect to the resistive heaters. The circuitry 640 and/or 645 will ensure the temperature is suitable for a current stage of DNA synthesis and will vary the temperature corresponding to each stage in the synthesis process. In one example, circuitry 645 may include processing circuitry for performing the functions of one or more of the reagent injection controller 135, microvalve controller 142 and temperature controller 160. Such controllers may also be implemented via other circuitry coupled to circuitry 640, 645.

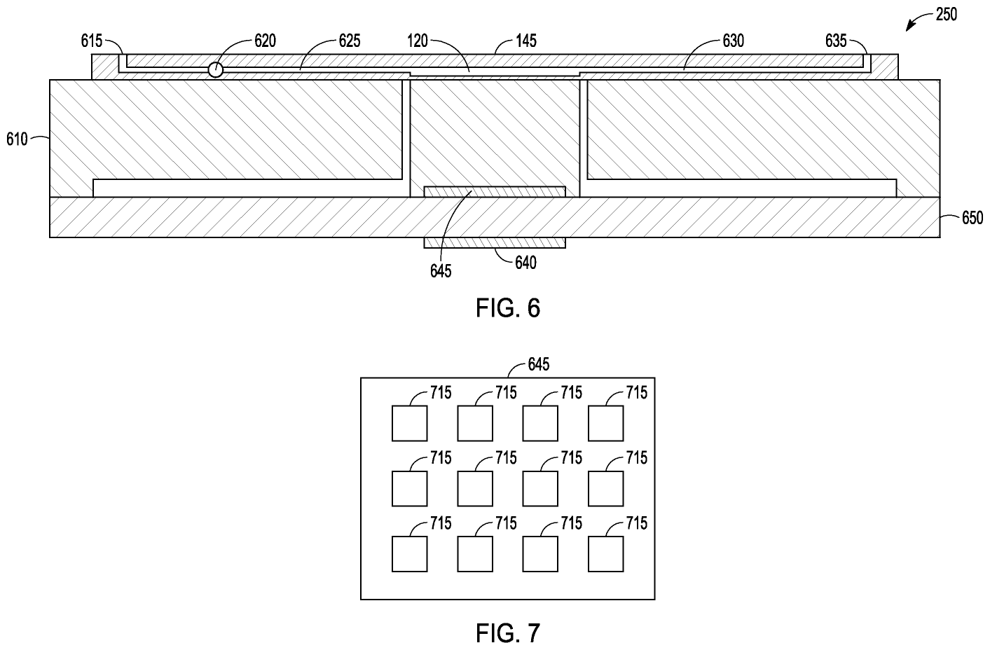

FIG. 7 is an example of positions of temperatures sensors 710 and heaters 715 on circuitry 645. In one example two temperature sensors 710 are provided for redundancy and are centered on the circuitry 645. As shown in this example, there are three rows and four columns of sensors/heaters. Various other configurations may be utilized that are suitable for both heating the substrate 610 and sensing the temperature of the substrate 610. For example, the temperature sensors may be positioned to one side or even separated into different positions, transposed with the heater positions. The temperatures sensors may be separated from each other rather than adjacent to each other as shown. In one example, the temperature sensors may be positioned on circuitry 640, which is located on a bottom side of the PCB 650.

In one example, the reaction chamber 120 may have a design point based on a solution volume of 20 ul. Given that design point, the reaction chamber 120 may have a height of approximately 1.70 mm for three main reasons: (a) cylindrical or oval chamber may help ensure that the initiator particles are concentrated in the central region of the reaction chamber; and the height of 1.70 mm to (b) guarantee the volume described above (and avoid waste of reagents), and (c) prevent the PDMS (polydimethylsiloxane), responsible for sealing the reaction site being sealed by oxygen plasma, from entering the chamber. This would result in an absorption of reagents and decrease in useful volume of the reaction site. In one example, the reaction chamber as a width of approximately 2.76 mm and a length of approximately 6.7 mm.

In one example, input channel width may be 0.3 mm and the output channel may have a width of 0.50 mm. Dimensions of all components may vary based on different design points, including fluid flow rates, reaction chamber size, and initiator particle size.

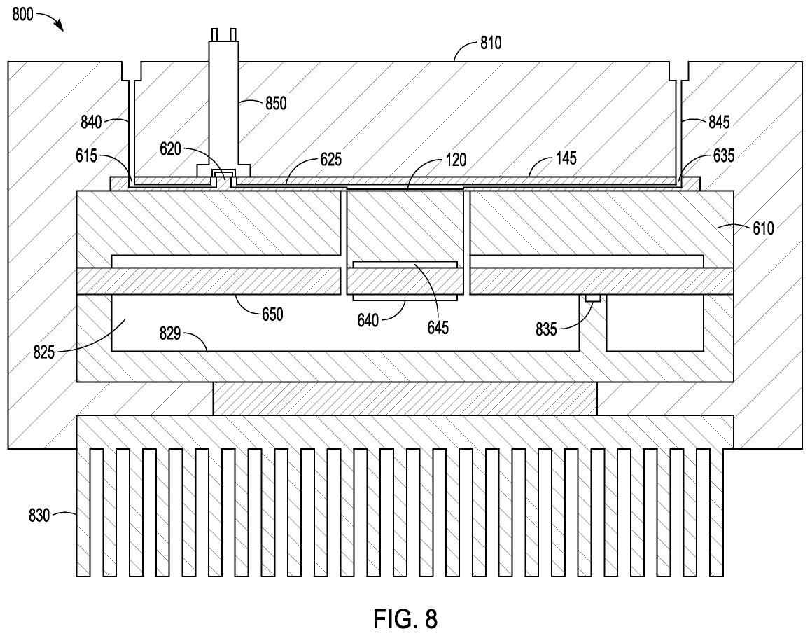

FIG. 8 is a cross section representation of an example synthesizer system 800 that includes addition structures to facilitate DNA synthesis. System 800 encapsulates the substrate 610, PCB 650, and microfluidic plate 145 that includes reaction chamber 120 as shown in FIG. 6 with an additional isolation layer 810. Isolation layer 810 serves to both support the microfluidic plate and additional components and thermally isolate them from ambient conditions.

In system 800, the substrate 610 extends below the PCB 650 as shown at bottom layer 820, providing a chamber 825 to provide a thermal buffer for processing circuitry supported by PCB 650. Heat exchanging fins 830 may also be provided to facilitate faster temperature control, such as reducing temperatures, for the reaction chamber 120. Fins 830 may be part of the substrate 610 in one example or otherwise formed of a thermally conductive material.

In one example, the bottom layer 820 includes a projection 835 that extends upward to contact the PCB 650. A further temperature sensor 845 may be positioned thermally proximate to the reaction chamber 120 and is laterally spaced from the heaters in circuitry 645 to provide temperature information representing the temperature of PCB 650. Such temperature information may be used by temperature controller 160 to ensure circuitry on PCB 650 is not subjected to temperatures outside a desired range.

The isolation layer 810 extends above the microfluidic plate 145 and includes channels 840 and 845 to reach the inlet 615 and the output 635 of the microfluidic plate 145. Further details of valve 620 are also shown at 850, extending through the isolation layer.

In further examples, the reaction chamber 120 can be a channel, a well, a cartridge, a pore, or reaction site. DNA synthesis devices can include as few as one reaction chamber that includes one or more initiator particles. In other examples, the DNA synthesis device can include any plural number of reaction chambers at least some of which or all can include initiator particles.

In one example, the DNA synthesis device can include 1 to 500 reaction chambers. Where multiple reaction chambers are present, each reaction chamber can have substantially the same dimensions. Alternatively, at least two reaction chambers may differ in their dimensions. For example, one reaction chamber may be shaped as a well, whereas another reaction chamber may be formed as a channel.

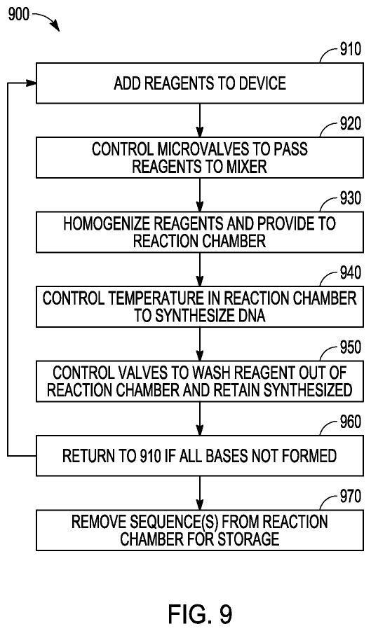

FIG. 9 is a flowchart illustrating a method 900 of synthesizing nucleic acid sequences utilizing synthesizer 100 and generally following the process 125. Method 900 begins at operation 910 by adding enzymatic synthesis reagents for adding a nucleic acid base to a nucleic acid sequence. The reagents are added to the synthesizer 100 via entry points, such as chambers 210 and 215, and include microparticle beads having initiator particles. Operation 920 controls microvalves to pass the reagents to the mixer 115. The mixer 115 homogenizes the reagents and provides the reagents to the reaction chamber 120 at operation 930. Operation 940 controls the temperature of the reaction chamber via a temperature control plate thermally coupled to the reaction chamber to achieve a temperature in the reaction chamber conducive to adding the nucleic acid base and synthesize DNA. Operation 950 controls valves to wash the enzymatic synthesis reagents from the reaction chamber while ensuring the beads containing synthesized DNA are retained in the reaction chamber 120. Fluid to wash the reagents may also be introduced via chambers 210 or 215. Following operation 950, a new cycle may be performed as indicated at 960, adding new reagents at operation 910 and performing operations 920-950 iteratively in order to increase the length of the synthesized DNA. Operation 970 removes the synthesized nucleic acid sequences from the reaction chamber for storing.

Method 900 may be performed by one or more controllers operating together, or a single controller suitably programmed to control reagent flow and reaction chamber temperature for formation of desired nucleic acid sequences.

Multiple DNA molecules representing a desired sequence may be produced utilizing the synthesizer system using method 900. The multiplicity of molecules produced can be in the ranges of 10's, 100's, 1000's, millions or even billions of copies of DNA molecules for each desired sequence. All of these copies representing all the desired sequences may be pooled into one master pool of molecules. It is typical of DNA writing systems that the writing may not be perfect, and if N molecules are synthesized to represent a given input sequence, not all of these will actually realize the desired sequence. For example, they may contain erroneous deletions, insertions, or incorrect or physically damaged bases.

The sequence can be determined by an encoder/decoder, which includes an algorithm with two functions: the encoder portion translates given digital data encoded format (e.g., digital/binary information) into a specific set of DNA sequences that are inputs to the DNA writer. The decoder portion translates a given set of DNA sequences of the type provided by the DNA reader, back into digital information.

The nucleotide oligomer can have bases chosen from adenine, cytosine, guanine, and thymine. In general, in order to reduce errors, the digital data encoding/decoding algorithm can comprise error detecting and error correcting codes selected to minimize error production, given the actual error modes of the DNA writer and DNA reader. These codes can be devised with the benefit of prior knowledge of the error modes, i.e., the propensity for particular errors of the writer and reader.

In various aspects, the error correcting codes reside within a single nucleotide sequence. For example, one segment of binary data is encoded in one DNA sequence, with the use of error correction and/or detection schemes on the DNA side. Such schemes may also involve encoding one segment of binary data into multiple DNA sequences, to provide another level of redundant encoding of information, which is analogous to error correction through redundant storage. Error detection schemes include, but are not limited to, repetition code, parity bits, checksums, cyclic redundancy checks, cryptographic hash functions, and error correcting codes such as hamming codes. Error correction schemes include, but are not limited to, automatic repeat request, error correcting code such as convolutional codes and block codes, hybrid automatic repeat request, and Reed-Solomon codes.

In various aspects, a method of devising an optimal or highly efficient error correcting encoding, wherein the incoming digital data is considered as binary words of length N, comprises the steps of: providing a space of all DNA words of length M, such that there are many more possible DNA words than binary words (i.e., 4^{M}>>2^{N}); and selecting a subset of 2^{N} of the DNA words to use as code words for encoding the 2^{N} binary information words, such that when each of these DNA code words is expanded into the set of probable DNA writing errors for the given word, and then that set further expanded by the set of probable reading errors words, these resulting 2^{N} sets of DNA words remain disjoint with high probability. In such a case, any word read by the reader can be properly associated back to the ideal encoded DNA word with very high probability. This method constitutes a combination of error correcting and error avoiding encoding of information. In addition, the decoding algorithm would also naturally make use of confidence or odds information supplied by the reader, to select the maximum likelihood/highest confidence decoding relative the encoding scheme.

Following synthesis of the coded oligomer, the coded oligomer can be cleaved from initiator particle. This can be accomplished by cleaving the bond between initiator particle and the coded oligomer. Following cleaving, initiator particle remains in the reaction chamber 120 and can be reused after being coupled to an initiator in a subsequent DNA synthesis procedure.

One consideration in optimizing the DNA data storage system costs is the time required to write data. For example, the critical time cost in many aspects may be the time cost of writing the data. In various aspects, the writing of certain slow-to-synthesize bases and sequence motifs are avoided in order to shorten the overall writing time. In other aspects, the writing is faster, such as by reducing the time spent on each chemistry cycle of some cyclical process that writes one base in many parallel synthesis reactions, with acceptance of a higher overall writing error rate. Process 125 may utilize different times and temperatures for extension, washing, and deprotection based on the bases to be synthesized.

Similarly, for reading, a faster reading process may be employed, with the trade-off being a higher rate of reading errors. In various examples, a faster reading process is employed without an increase in error by avoiding the introduction of certain types of sequences in the encoding that are difficult to read at a rapid rate, such as homopolymer runs. In either case, the information encoding/decoding algorithm can be co-optimized with these choices that allow for faster reading/writing but with extra error modes to be avoided, or avoiding slow-to-read/write sequence motifs, handled within the encoding/decoding.

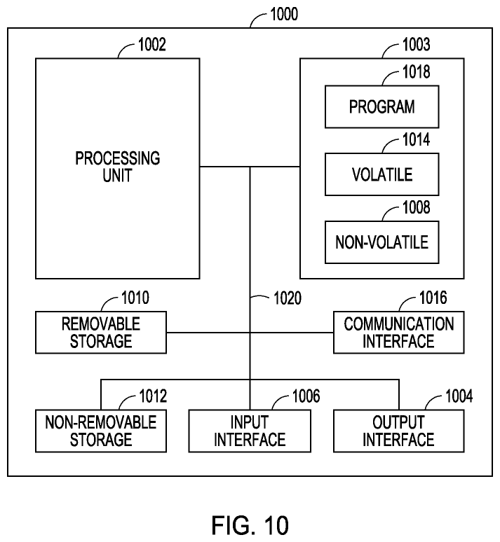

FIG. 10 is a block schematic diagram of a computer system 1000 to control the synthesizer to generate DNA sequences according to example embodiments. All components need not be used in various embodiments.

One example computing device in the form of a computer 1000 may include a processing unit 1002, memory 1003, removable storage 1010, and non-removable storage 1012. Although the example computing device is illustrated and described as computer 1000, the computing device may be in different forms in different embodiments.

Although the various data storage elements are illustrated as part of the computer 1000, the storage may also or alternatively include cloud-based storage accessible via a network, such as the Internet or server-based storage. Note also that an SSD may include a processor on which the parser may be run, allowing transfer of parsed, filtered data through I/O channels between the SSD and main memory.

Memory 1003 may include volatile memory 1014 and non-volatile memory 1008. Computer 1000 may include—or have access to a computing environment that includes—a variety of computer-readable media, such as volatile memory 1014 and non-volatile memory 1008, removable storage 1010 and non-removable storage 1012. Computer storage includes random access memory (RAM), read only memory (ROM), erasable programmable read-only memory (EPROM) or electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage or other magnetic storage devices, or any other medium capable of storing computer-readable instructions.

Computer 1000 may include or have access to a computing environment that includes input interface 1006, output interface 1004, and a communication interface 1016. Output interface 1004 may include a display device, such as a touchscreen, that also may serve as an input device. The input interface 1006 may include one or more of a touchscreen, touchpad, mouse, keyboard, camera, one or more device-specific buttons, one or more sensors integrated within or coupled via wired or wireless data connections to the computer 1000, and other input devices. The computer may operate in a networked environment using a communication connection to connect to one or more remote computers, such as database servers. The remote computer may include a personal computer (PC), server, router, network PC, a peer device or other common data flow network switch, or the like. The communication connection may include a Local Area Network (LAN), a Wide Area Network (WAN), cellular, Wi-Fi, Bluetooth, or other networks. According to one embodiment, the various components of computer 1000 are connected with a system bus 1020.

Computer-readable instructions stored on a computer-readable medium are executable by the processing unit 1002 of the computer 1000, such as a program 1018. The program 1018 in some embodiments comprises software to implement one or more methods described herein. A hard drive, CD-ROM, and RAM are some examples of articles including a non-transitory computer-readable medium such as a storage device. The terms computer-readable medium, machine readable medium, and storage device do not include carrier waves or signals to the extent carrier waves and signals are deemed too transitory. Storage can also include networked storage, such as a storage area network (SAN). Computer program 1018 along with the workspace manager 1022 may be used to cause processing unit 1002 to perform one or more methods or algorithms described herein.

### EXAMPLES

1. A microfluidic system for performing nucleic acid synthesis incudes a microfluidic plate having a reaction chamber coupled to a microfluidic plate input and a microfluidic plate output, a temperature control plate thermally coupled to the microfluidic plate, a reagent injection plate coupled to receive enzymatic synthesis reagents, a microvalve plate coupled between the reagent injection plate and the microfluidic plate input, and a controller coupled the temperature control plate, and the microvalve plate to control the microfluidic system to controllably synthesize nucleic acid sequences.
2. The system of claim 1 and further including a temperature sensor thermally coupled to the microfluidic plate.
3. The system of example 2 and further including a temperature controller coupled to receive temperature information from the temperature sensor and to control the temperature control plate.
4. The system of example 3 wherein the temperature control plate includes heaters that are controlled by the controller using a proportional/integral/derivative (PID) algorithm.
5. The system of example 4 wherein the controller controls the temperature for extending nucleic acid sequences to −5° C. and for deprotection to 120° C.
6. The system of example 4 wherein the controller controls the temperature for extending nucleic acid sequences to 4° C. and for deprotection to 100° C.
7. The system of any of examples 1-6 wherein the microfluidic plate and temperature control plate are vertically stacked and wherein the microfluidic plate is formed of ceramic.
8. The system of any of examples 1-7 wherein the temperature control plate includes at least one resistive heater and at least one temperature sensor.
9. The system of example 8 wherein the temperature control plate includes a first board supporting the at least one resistive heater and at least one temperature sensor.
10. The system of any of examples 1-9 wherein the controller is configured to control reagent flow and temperature for forming data encoded nucleic acid sequences.
11. The system of any of examples 1-10 and further including a mixer coupled to mix reagents and provide mixed reagents to the reaction chamber wherein the mixer comprises a serpentine channel that includes orthogonal pairs of extensions to promote vortex flow within the extensions.
12. The system of any of examples 1-11 wherein the reaction chamber has a surface comprising gold.
13. The system of any of examples 1-12 and further including an initiator particle input coupled to the reaction chamber for providing microparticle beads having gold initiator particles.
14. The system of any of examples 1-13 wherein the reaction chamber is elliptical in shape having a long axis and a short axis.
15. The system of example 14 wherein the reaction chamber includes an input and a first output disposed on opposite sides of the long axis.
16. The system of example 15 and further including a filter coupled to the first output.
17. The system of any of examples 15-16 wherein the reaction chamber includes a second output radially spaced from the first output along the short axis.
19. The system of any of examples 1-17 wherein the reaction chamber is deeper in a middle portion of the reaction chamber than around a perimeter of the reaction chamber.
19. A microfluidic system for performing nucleic acid synthesis incudes a microfluidic plate having a reaction chamber having an input to receive enzymatic synthesis reagents, a reagent injection plate coupled to provide the enzymatic synthesis reagents to the reaction chamber, a microvalve plate coupled between the reagent injection plate and the microfluidic plate input, a temperature control plate thermally coupled to the microfluidic plate, and a controller coupled to the temperature control plate, and the microvalve plate to sequentially control the microvalve plate to provide selected enzymatic synthesis reagents and the temperature control plate to control the temperature of the reaction chamber to synthesize nucleic acid sequences.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “DNA” refers to both biological DNA molecules and synthetic versions, such as made by nucleotide phosphoramidite chemistry, ligation chemistry or other synthetic organic methodologies. DNA, as used herein, also refers to molecules comprising chemical modifications to the bases, sugar, and/or backbone, such as known to those skilled in nucleic acid biochemistry. These include, but are not limited to, methylated bases, adenylated bases, other epigenetically marked bases, thiol modified bases, and non-standard or universal bases such as inosine or 3-nitropyrrole, or other nucleotide analogues, or ribobases, or abasic sites, or damaged sites. DNA also refers expansively to DNA analogues such as peptide nucleic acids (PNA), locked nucleic acids (LNA), and the like, including the biochemically similar RNA molecule and its synthetic and modified forms. All these biochemically closely related forms are implied by the use of the term DNA, in the context of the data storage molecule used in a DNA data storage system herein. Further, the term DNA herein includes single stranded forms, double helix or double-stranded forms, hybrid duplex forms, forms containing mismatched or non-standard base pairings, non-standard helical forms such as triplex forms, and molecules that are partially double stranded, such as a single-stranded DNA bound to an oligo primer, or a molecule with a hairpin secondary structure. Generally as used herein, the term DNA refers to a molecule comprising a single-stranded component that can act as the template for a polymerase enzyme to synthesize a complementary strand therefrom.

DNA sequences as written herein, such as GATTACA, refer to DNA in the 5′ to 3′ orientation, unless specified otherwise. For example, GATTACA as written herein represents the single stranded DNA molecule 5′-G-A-T-T-A-C-A-3′. In general, the convention used herein follows the standard convention for written DNA sequences used in the field of molecular biology.

The term “polymerase” refers to an enzyme that catalyzes the formation of a nucleotide chain by incorporating DNA or DNA analogues, or RNA or RNA analogues, against a template DNA or RNA strand. The term polymerase includes, but is not limited to, wild-type and mutant forms of DNA polymerases, such as Klenow, E. Coli Pol I, Bst, Taq, Phi29, and T7, wild-type and mutant forms of RNA polymerases, such as T7 and RNA Pol I, and wild-type and mutant reverse transcriptases that operate on an RNA template to produce DNA, such as AMV and MMLV.

The term “dNTP” refers to both the standard, naturally occurring nucleoside triphosphates used in biosynthesis of DNA (i.e., dATP, dCTP, dGTP, and dTTP), and natural or synthetic analogues or modified forms of these, including those that carry base modifications, sugar modifications, or phosphate group modifications, such as an alpha-thiol modification or gamma phosphate modifications, or the tetra-, penta-, hexa- or longer phosphate chain forms, or any of the aforementioned with additional groups conjugated to any of the phosphates, such as the beta, gamma or higher order phosphates in the chain. In general, as used herein, “dNTP” refers to any nucleoside triphosphate analogue or modified form that can be incorporated by a polymerase enzyme as it extends a primer, or that would enter the active pocket of such an enzyme and engage transiently as a trial candidate for incorporation.

The terms, “binary data” or “digital data” refers to data encoded using the standard binary code, or a base 2 {0,1}alphabet, data encoded using a hexadecimal base 16 alphabet, data encoded using the base 10 {0-9}alphabet, data encoded using ASCII characters, or data encoded using any other discrete alphabet of symbols or characters in a linear encoding fashion.

The term, “digital data encoded format” refers to a series of binary digits, or other symbolic digits or characters that come from the primary translation of DNA sequence features used to encode information in DNA, or the equivalent logical string of such classified DNA features. In some aspects, information to be archived as DNA may be translated into binary, or may exist initially as binary data, and then this data may be further encoded with error correction and assembly information, into the format that is directly translated into the code provided by the distinguishable DNA sequence features. This latter association is the primary encoding format of the information. Application of the assembly and error correction procedures is a further, secondary level of decoding, back towards recovering the source information.

The term, “distinguishable DNA sequence features” means those features of a data-encoding DNA molecule that, when processed by a sensor polymerase, produce distinct signals that can be used to encode information. Such features may be, for example, different bases, different modified bases or base analogues, different sequences or sequence motifs, or combinations of such to achieve features that produce distinguishable signals when processed by a sensor polymerase.

The term, a “DNA sequence motif” refers to both a specific letter sequence or a pattern representing any member of a specific set of such letter sequences. For example, the following are sequence motifs that are specific letter sequences: GATTACA, TAC, or C. In contrast, the following are sequence motifs that are patterns: G[A/T]A is a pattern representing the explicit set of sequences {GAA, GTA}, and G[2-5] is a pattern referring to the set of sequences {GG, GGG, GGGG, GGGGG}. The explicit set of sequences in the unambiguous description of the motif, while such pattern shorthand notations as those are common compact ways of describing such sets. Motif sequences such as these may be describing native DNA bases, or may be describing modified bases, in various contexts. In various contexts, the motif sequences may be describing the sequence of a template DNA molecule, and/or may be describing the sequence on the molecule that complements the template.

The term, a “data-encoding DNA molecule,” or “DNA data encoding molecule,” refers to a molecule synthesized to encode data in DNA, or copies or other DNA derived from such molecules.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A microfluidic system for performing nucleic acid synthesis comprising:
a microfluidic plate having a reaction chamber coupled to a microfluidic plate input and a microfluidic plate output;
a temperature control plate thermally coupled to the microfluidic plate;
a reagent injection plate coupled to receive enzymatic synthesis reagents;
a microvalve plate coupled between the reagent injection plate and the microfluidic plate input; and
a controller coupled the temperature control plate, and the microvalve plate to control the microfluidic system to controllably synthesize nucleic acid sequences.

2. The system of claim 1 and further comprising a temperature sensor thermally coupled to the microfluidic plate.

3. The system of claim 2 and further comprising a temperature controller coupled to receive temperature information from the temperature sensor and to control the temperature control plate.

4. The system of claim 3 wherein the temperature control plate includes heaters that are controlled by the controller using a proportional/integral/derivative (PID) algorithm.

5. The system of claim 4 wherein the controller controls the temperature for extending nucleic acid sequences to −5° C. and for deprotection to 120° C.

6. The system of claim 4 wherein the controller controls the temperature for extending nucleic acid sequences to 4° C. and for deprotection to 100° C.

7. The system of claim 1 wherein the microfluidic plate and temperature control plate are vertically stacked and wherein the microfluidic plate is formed of ceramic.

8. The system of claim 1 wherein the temperature control plate comprises at least one resistive heater and at least one temperature sensor.

9. The system of claim 8 wherein the temperature control plate comprises a first board supporting the at least one resistive heater and at least one temperature sensor.

10. The system of claim 1 wherein the controller is configured to control reagent flow and temperature for forming data encoded nucleic acid sequences.

11. The system of claim 1 and further comprising a mixer coupled to mix reagents and provide mixed reagents to the reaction chamber wherein the mixer comprises a serpentine channel that includes orthogonal pairs of extensions to promote vortex flow within the extensions.

12. The system of claim 1 wherein the reaction chamber has a surface comprising gold.

13. The system of claim 1 and further comprising an initiator particle input coupled to the reaction chamber for providing microparticle beads having gold initiator particles.

14. The system of claim 1 wherein the reaction chamber is elliptical in shape having a long axis and a short axis.

15. The system of claim 14 wherein the reaction chamber comprises an input and a first output disposed on opposite sides of the long axis.

16. The system of claim 15 wherein the reaction chamber comprises a second output radially spaced from the first output along the short axis.

17. The system of claim 15 and further comprising a filter coupled to the first output.

18. The system of claim 1 wherein the reaction chamber is deeper in a middle portion of the reaction chamber than around a perimeter of the reaction chamber.

19. A microfluidic system for performing nucleic acid synthesis comprising:
a microfluidic plate having a reaction chamber having an input to receive enzymatic synthesis reagents;
a reagent injection plate coupled to provide the enzymatic synthesis reagents to the reaction chamber;
a microvalve plate coupled between the reagent injection plate and the microfluidic plate input;
a temperature control plate thermally coupled to the microfluidic plate; and
a controller coupled to the temperature control plate, and the microvalve plate to sequentially control the microvalve plate to provide selected enzymatic synthesis reagents and the temperature control plate to control the temperature of the reaction chamber to synthesize nucleic acid sequences.

20. A method of synthesizing nucleic acid sequences, the method comprising:
A. adding enzymatic synthesis reagents to a microfluidic device for adding a nucleic acid base to a nucleic acid sequence;
B. controlling valves of the microfluidic device to provide the selected enzymatic synthesis reagents to a mixing channel of the microfluidic device;
C. receiving homogenized enzymatic synthesis reagents in a reaction chamber of the microfluidic device;
D. controlling temperature of the reaction chamber via a temperature control plate thermally coupled to the reaction chamber to achieve a temperature in the reaction chamber conducive to adding the nucleic acid base;
E. controlling valves of the microfluidic device to wash the enzymatic synthesis reagents from the reaction chamber while retaining synthesized DNA in the reaction chamber; and
F. repeating steps A, B, C, D, and E for multiple different combination of enzymatic synthesis reagents to form bases representative of encoded data.
